(19) United States
(12) Patent Application Publication
(10) Pub. No.: US 2010/0263675 A1
(43) Pub. Date: Oct. 21, 2010

(54) Condom with Localized Active Agent

(75) Inventors: Beng Sim Chuah, Petaling Jaya (MY); Tsui Shih Yeun, Puchong (MY); David M. Lucas, Petaling Jaya (MY); Burton Van Rooyen, East Malvern (AU); Michael S. Zedalis, Mendham, NJ (US)

Correspondence Address: DIEHL SERVILLA LLC, 33 WOOD AVE SOUTH, SECOND FLOOR, SUITE 210, ISELIN, NJ, 08830, US

(73) Assignee: Ansell Healthcare Products LLC, Red Bank, NJ (US)

(21) Appl. No.: 12/427,061

(22) Filed: Apr. 21, 2009

(51) Int. Cl. A61F 6/04

(52) U.S. Cl. 128/844

(57) ABSTRACT

Provided are condoms and methods of making and using the same, the condoms having an elastomeric layer and a low melt wax bead which comprises an active ingredient mixed therein, wherein the bead is affixed on the interior surface of the elastomeric layer in the tip of the condom. Active ingredients can include spermicides such as nonoxynol-9 and vasodilators such as niacin, sildenafil citrate, and nitroglycerin and male desensitizing agents such as benzocaine. The bead can comprise a natural wax in combination with polyethylene glycol or it can be a polyethylene glycol-based wax. The exterior surface of the elastomeric layer is substantially free of the active ingredient.

### TECHNICAL FIELD

Embodiments of the present invention relate to condoms having an active ingredient localized in the closed end of a condom. Specifically provided are condoms having one or more active ingredients solubilized or mixed as a fine water soluble powder within a low melting wax bead. Ingredients, for example, include liquid nonoxynol-9 and other spermicides, as well as niacin, nitroglycerin, and sildenafil citrate, and other vasodilators.

### BACKGROUND

Condoms and other prophylactics and protective devices provide physical barriers against the transmission of bodily fluids or other fluids. Chemical barriers can be also used alone or in conjunction with condoms to prevent such transmission. In some instances, the chemical barriers serve as a supplemental form of protection in the event the physical barrier is breached. U.S. Pat. No. 5,284,158 (Mallette) provides a sensitive condom that locates a spermicide in a latex cap of a condom as well as in a space between the main sheath of the condom and a reinforcing membrane. The packaging of Mallette, moreover, includes the spermicide to prevent drying of the membrane. In U.S. Pat. No. 4,795,425 (Pugh), a double dosage of spermicide is provided, where a first dosage is provided in a semi-solid state in the closed end of the condom and a second dosage is provided on the outside surface of the condom. The semi-solid dosage is provided as a cream or ointment where the spermicide is mixed with forms of polyethylene glycol, including Carbowax 400, which is not a solid wax at room temperature, and Carbowax 3350, which is solid and has a melting point in the range of 54° C. to 57° C. Such a cream or ointment is stated to have a melting point in the range of 113-127° F. (45-53° C.).

Spermicides such as nonoxynol-9, however, have been identified as possible irritants to mucosal membranes. U.S. Pat. No. 5,387,611 (Rubinstein) provides non-irritating agents having anti-bacterial and anti-viral activity using butylurea in combination with nonoxynol-9 and/or benzalkonium chloride.

In addition to spermicides, other active ingredients can be delivered via condoms and other prophylactics and protective devices to treat, for example, sexual dysfunction. Topical application of therapeutic agents such as vasodilators is one option. As provided in U.S. Pat. Nos. 4,829,991 (Boeck) and 6,840,244 (Kemp), condoms can be used to deliver therapeutic agents such as vasodilators and erectogenic compounds. In Kemp, an erectogenic compound is immobilized in the closed end of a condom by the use of a solvent carrier, in the optional presence of a film-forming agent, which is subsequently evaporated to leave the erectogenic compound in a finely-divided form or as a film layer. The erectogenic compound (or composition) of Kemp is immiscible with a lubricant of the condom. An erectogenic composition can comprise an erectogenic compound dispersed or dissolved in a gel carrier comprising a liquid medium and a thickening agent. Desired thickeners depend on the liquid medium. For a water-based lubricant, a vegetable oil liquid medium is preferred, thickeners for which include hectorite, jojoba oils, and waxes. For an oil-based lubricant, a polyhydroxy-based medium is preferred, thickeners for which include cellulose derivatives. This erectogenic compound dispersed or dissolved in a gel carrier does not contain the erectogenic compound in a softenable or meltable wax at the closed end of a condom. In U.S. Pat. No. 7,086,403 (Harrison), a condom has a first lubricating composition having a relatively high viscosity and containing a male genitalia desensitizing agent located on the inside surface and a second lubricating composition having a lower viscosity on the outside surface.

Particles of wax can be used in coatings on elastomeric articles to aid in donning or blocking, as provided in, for example, U.S. Pat. Nos. 5,405,666 (Brindle); 6,075,081 (Nile); and 6,306,514 (Weikel). Such use of wax, however, does not provide localized delivery of an active ingredient.

There is a need, therefore, to provide active agents on condoms in convenient ways to reduce irritation to mucosal membranes by the dilution of agents delivered. There is a need for a condom having one or more localized ingredients disposed in the tip. There is also a need for localizing spermicidal and/or erectogenic and/or male desensitizing compounds on the inside of a condom to avoid contact of such to the female partner.

### SUMMARY

Provided are condoms and methods of making and using the same, the condoms having an elastomeric layer and a low melt wax bead which comprises an active ingredient mixed therein, wherein the bead is affixed on the interior surface of the elastomeric layer in the tip of the condom. In a detailed embodiment, a condom comprises an elastomeric layer and a low melt wax bead which comprises nonoxynol-9 mixed therein. Another detailed embodiment includes a male desensitizing agent mixed into the bead, for example benzocaine, dibucaine, lidocaine, tetracaine, and salts and derivatives thereof. The beads can comprise a polyethylene glycol-based wax of selected molecular weight having a melting point in the range of 25° C. to 40° C. Alternatively, the beads may be a solidified liquid solution of a natural wax such as beeswax, the melting point of which can be depressed by addition of polyethylene glycol of appropriate molecular weight in the liquid state. The bead of wax is affixed on the interior surface of the elastomeric layer in the tip of the condom and the exterior surface of the elastomeric layer is substantially free of the active ingredient, such as nonoxynol-9, niacin, nitroglycerin, sildenafil citrate, or benzocaine.

In an aspect, provided are methods for manufacturing a condom, the method comprising the steps of: providing a molten solution of a low melt wax containing an active ingredient; providing the condom; dropping a bead of the molten solution on the interior surface and in the tip of the condom; permitting the bead to reach ambient temperature and solidifying the bead such that the bead is localized on the interior surface and in the tip of the condom; and packaging the condom. The step of providing the molten solution can comprise melting the low melt wax and mixing the active ingredient having a melting point less than that of the low melt wax with the low melt wax to form a substantially uniform molten solution. Alternatively or additionally, such step can comprise melting the wax and adding a fine powder of water soluble active agent.

Further aspects include methods for providing a spermicide within the closed end of the condom, the methods comprising providing a condom which comprises an elastomeric layer and a low melt wax bead which comprises nonoxynol-9 mixed therein, the bead comprising a natural wax modified to have a melting point in the range of 25° C. to 40° C. and a polyethylene glycol polymer with an appropriate molecular weight to have a melting point in the range of 25° C. to 40° C., wherein the bead is affixed on the interior surface of the elastomeric layer in the tip of the condom and wherein the exterior surface of the elastomeric layer is substantially free of nonoxynol-9.

Further aspects include methods for providing an erectogenic compound within the closed end of the condom, the methods comprising providing a condom which comprises an elastomeric layer and a low melt wax bead which comprises an erectogenic compound mixed therein, the bead comprising a natural wax modified to have a melting point in the range of 25° C. to 40° C. and a polyethylene glycol polymer with an appropriate molecular weight to have a melting point in the range of 25° C. to 40° C., wherein the bead is affixed on the interior surface of the elastomeric layer in the tip of the condom and wherein the exterior surface of the elastomeric layer is substantially free of the erectogenic compound.

### DETAILED DESCRIPTION

It has been found that delivery of an active ingredient via a condom can be achieved by localizing the active ingredient using a bead of wax or other suitable carrier in the tip of the condom on its interior surface. In this way, the active ingredient can be delivered in a diluted, yet still effective form, to reduce irritation. Moreover, location of the active ingredient in the interior of the condom reduces exposure of the users' partner to the active ingredient. Use of a low melting wax, for example, permits substantially uniform distribution of the active ingredient incorporated in the bead to further aid in effective delivery of the ingredient.

In a first aspect, provided are condoms having an elastomeric layer and a low melt wax bead which comprises an active ingredient mixed therein, wherein the bead is affixed on the interior surface of the elastomeric layer in the tip of the condom. One or more embodiments provide that the exterior surface of the elastomeric layer is substantially free of the active ingredient. By “substantially free of the active ingredient” it is meant that the exterior surface of the elastomeric layer is not provided with the active ingredient nor is there a significant amount of active ingredient on the exterior surface. It is recognized that as a result of the handling of the condom miniscule amounts of active ingredient may inadvertently reside on the exterior surface. For the purpose of the present invention, active ingredients that are not intentionally located on the exterior surface are considered insignificant.

Reference to “low melt” refers to a material, such as wax or other suitable carrier that softens, flattens, and/or melts in the presence of body heat, which is approximately 37° C. In one embodiment, the bead softens in the presence of body heat thereby substantially exposing the active ingredient. In one or more embodiments, the wax or other suitable carrier melts in the range of 25° C.-40° C.

One embodiment provides that the active ingredient comprises a spermicide. In a specific embodiment, the spermicide comprises nonoxynol-9. Other spermicides include oxtoxynol-9, menfegol, a vaginal contraceptive compound with a surface active agent p-menthanylphenyl polyoxyethylene ether, lactic acid, neem oil, membrane disruptive agents, derivatives thereof, and combinations thereof.

Another embodiment provides that the active ingredient comprises a vasodilator. A specific embodiment provides that the vasodilator comprises a niacin component. A niacin component includes niacin (also known as nicotinic acid), nicotinamide, methyl nicotinate (niacin methyl ester), derivatives thereof, or combinations thereof.

The terms “erectogenic compound” and “vasodilator,” as used herein, refers to a substance which causes vasodilation, enhances blood circulation to body parts, increases nitrous oxide levels, general heat or create the feeling of heat and/or increase sexual arousal, response or otherwise treat sexual dysfunction. Such substances should be absorbed readily into the skin and should not react adversely with any of the other ingredients, capsules and/or films used in some embodiments of the present invention. Embodiments of the present invention can utilize selective inhibitors of phosphodiesterase, for example, cyclic guanosine monophosphate (cGMP) specific phosphodiesterase type 5 (“PDE-5”). PDE-5 inhibitors include, but are not limited to, sildenafil, tadalafil, vardenafil, derivatives, and pharmaceutically acceptable salts thereof. Other suitable vasodilators include prostaglandin compounds, for example prostaglandin E-1, which is known under the pharmaceutical name of alprostadil. Another suitable ingredient is testosterone.

Other embodiments may include compounds which have vasodilatory effects which include niacin, L-arginine, ginger, menthol, menthyl lactate, and other menthol derivatives and precursors, compounds such as peppermint, spearmint and other mint oils, nitric oxide, precursors of nitric oxide, i.e., glyceryl trinitrate (nitroglycerin), organic nitrites, i.e., amyl nitrite, isoamyl nitrite and the like, and variations thereof, while other embodiments utilize other vasodilators known in the art.

In some embodiments, the elastomeric layer is comprised of natural rubber latex, synthetic polyisoprene, guayule, polyurethane, copolymers or combinations thereof. The term “natural rubber latex” as used in this disclosure encompasses cured elastomeric material sourced from Hevea brasiliensis (the traditional rubber tree), Parthenium argentatum (guayule), sunflower, goldenrod and the like, as well as genetically modified variations of these or other biological sources.

Other embodiments utilize other substances to form the elastomeric layer that are known in the art.

The bead may include waxes that are either synthetic or natural. The natural waxes that may be generally used include montan, carnauba, bees wax, bayberry-myrtle, candelialla, caranday, castor bean, asparto-grass, Japan, ouricury, retamo-ceri, mimbi, schlack, spermaceti, sugar-cane, and wool lanolin. Synthetic waxes generally include polyethylene and modified polyethylenes, polypropylene and modified polypropylenes, and hydrogen-based materials. If the melting point is greater than the 25° C. to 40° C. range, the wax may be melted and a low melting wax may such as polyethylene glycol wax with a suitable low molecular weight and corresponding low melting point may be added to the wax to form a melt solution and frozen to produce a wax with a melting point in the range of 25° C. to 40° C.

The bead can comprise polyethylene glycol (PEG). One exemplary PEG is PEG 600 with a melting point of 20-25° C. (68-77° F.). Another potential PEG is PEG1500, which melts at 44-48° C. (111-118° F.), which when mixed with lower melting materials can be suitable for the bead. Thus, in one or more embodiments, the bead comprises one or more polyethylene glycol polymers having an overall molecular weight in the range of 400 to 1500 (or 600 to 1300, or even 800-1100) and having a melting point in the range of 25° C. to 40° C. (or even 36-39° C.). An additional PEG is PEG 1000, which has a melting point of 38° C., which is suited for localized incorporation of active ingredients of the present invention. A polyethylene glycol-based wax containing PEG-6 and PEG-32, which has a molecular weight in the range of 470-530 g/mol, may also be suitable. With the PEG-containing bead, it is expected that upon contact skin-generated moisture, warming occurs due to an exothermic heat of dissolution on the order of an increase in temperature of about 5-7° C. The warming effect can help enlarge skin pores and enhance penetration of the active ingredient.

One or more embodiments provide that the exterior surface of the condom contains a lubricant. This lubricant can contain silicone or any other lubricating material that is compatible and not miscible with the wax bead. With reference to compatible, it is meant that the lubricating material would not destabilize the wax bead or cause the active agent to be released if the lubricating material contacts the wax bead. Suitable lubricants can include silicone oils such as dimethicone, dimethylsiloxane, cyclomethicone and other silicone derivatives and other non-PEG wax solubilizing materials. This lubricant can further contain materials that enhance the sexual experience for the female and/or alleviate symptoms associated with female sexual dysfunction. Such materials include, but are not limited to: L-arginine; menthol, menthyl lactate, and other menthol derivatives and precursors; a prostaglandin compound, such as prostaglandin E-1, which is known under the pharmaceutical name of alprostadil. Another possible addition to the lubricant is an astringent such as witch hazel.

One or more embodiments provide that the interior surface of the condom further contains a lubricant that is free of the active agent. This lubricant can contain silicone or any other lubricating material that is compatible and not miscible with the wax bead. If a lubricant on the exterior surface is provided, then typically the lubricant on the interior surface is of a higher viscosity that that on the exterior surface.

In a detailed aspect, provided are condoms comprising an elastomeric layer and a low melt wax bead which comprises nonoxynol-9 mixed therein, the bead comprising a modified natural wax and a polyethylene glycol polymer wax with appropriate molecular weight and melting point, wherein the bead is affixed on the interior surface of the elastomeric layer in the tip of the condom and wherein the exterior surface of the elastomeric layer is substantially free of nonoxynol-9.

In one or more embodiments, the bead nonoxynol-9 mixed therein is present in an amount in the range of 10 to about 100 mg. The wax bead may be up to 1 gram or more. It may be desirable to provide the active ingredient in an amount in the range of 1 to 1½ the dosage needed, rather than an excess of 3 times or more of the required dosage, which is the amount of nonoxynol-9 generally needed in a water based or silicone based lubricant.

Further aspects provide methods for manufacturing a condom, the methods comprising the steps of: melting a low melt wax; mixing an active ingredient in the form of a molten liquid or as a fine dispersion of a water soluble powder with the low melt wax to form a substantially uniform mixture; providing the condom; dropping a bead of the mixture on the interior surface and in the tip of the condom; permitting the bead to reach ambient temperature such that the bead is solidified and localizes the active ingredient on the interior surface and in the tip of the condom; and packaging the condom.

Additional aspects include methods for providing a spermicide in the closed end of a condom, the methods comprising providing a condom which comprises an elastomeric layer and a low melt wax bead which comprises nonoxynol-9 mixed therein, the bead comprising a modified natural wax and a polyethylene glycol polymer wax with appropriate molecular weight and melting point, wherein the bead is affixed on the interior surface of the elastomeric layer in the tip of the condom and wherein the exterior surface of the elastomeric layer is substantially free of nonoxynol-9.

Further embodiments of the present invention include a packaging containing the condom, as otherwise described herein. Examples of packaging include, without limitation, wrappers, boxes, tubs having a sealed enclosure and combinations thereof.

At least one embodiment of the present invention includes the step of packaging the finished condom. Specifically, in some embodiments, the formed elastomeric layer having localized bead thereon is packaged in a wrapper, box, tub with sealed enclosure or any combination thereof. At least one embodiment of a method of manufacturing a condom of the present invention includes the step of packaging the finished condom. Specifically, in some embodiments, the formed elastomeric layer having at least one ingredient disposed thereon is packaged in a wrapper, box, tub with sealed enclosure, or any combination thereof. In some embodiments, the materials used in forming the package are selected from materials that can form packages that are substantially impermeable to the passage of air and moisture. The packaging materials include, but are not limited to, cellulosic materials, such as paper or paperboard; polymeric films and sheeting; metallic foils; and composite materials formed from two or more of these materials. The packages may be sealed by bonding processes including, but not limited to, adhesive, heat, ultrasonic welding, pressure and combinations of these bonding processes.

Processes that can be useful in making condoms in accordance with various aspects of the present invention are well known to those of ordinary skill in the art.

### EXAMPLES

Examples of condoms according to embodiments of the invention are shown below. These and other exemplary ingredients may be present in larger or smaller quantities on one or more surfaces of the condom for particular applications and are as follows:

### Example 1A

A modified natural wax and polyethylene glycol polymer wax with appropriate molecular weight and melting point formulation is melted to bring the wax few degrees (˜1-10° C.) above the melting point, to which 100 mg nonoxynol-9 liquid is added to form a mixture. A bead of the mixture is dropped into the tip of a condom on the interior surface and is allowed to reach ambient temperature and solidify the wax bead. The condom is then packaged. Nonoxynol-9 can be dosed up to 15 to 20% of the weight of the wax. Use of higher concentration of nonoxynol-9 may lead to segregation of nonoxynol-9 within the wax bead resulting in possible skin irritation or even potentially localized weakening of the condom. A 0.5 gram wax bead could contain 75 mg of nonoxynol-9.

### Example 1B

A raw material having a mixture of polyethylene glycol waxes, PEG-6 and PEG-32 is sold under the product name Lanogen 1500 by Clariant, was melted to bring the wax few degrees (˜1-10° C.) above the melting point, to which 20% nonoxynol-9 liquid was added to form a homogenized wax mixture without separation A bead of the mixture was dropped into the tip of a condom on the interior surface and was allowed to reach ambient temperature and solidify the wax bead.

### Example 2

A modified natural wax and polyethylene glycol polymer wax with appropriate molecular weight and melting point formulation is melted to bring the wax few degrees (˜1-10° C.) above the melting point, to which 100 mg nitroglycerin liquid is added to form a mixture. A bead of the mixture is dropped into the tip of a condom on the interior surface and is allowed to reach ambient temperature and solidify the wax bead. The condom is then packaged.

### Example 3A

A polyethylene glycol polymer wax (PEG 1000) with appropriate molecular weight and melting point formulation is melted to bring the wax few degrees (˜1-10° C.) above the melting point, to which 100 mg of finely powdered water-soluble niacin is added to form a mixture. A bead of the mixture is dropped into the tip of a condom on the interior surface and is allowed to reach ambient temperature and solidify the wax bead. The condom is then packaged. A 1 gram wax bead may contain 5% niacin resulting in 50 mg of niacin.

### Example 3B

A polyethylene glycol polymer wax mixed with glycerin and containing 0.25% menthol was heated, to which 20 mg of finely powdered water-soluble niacin was added to form a mixture. A bead of the mixture was dropped into the tip of a condom on the interior surface and was allowed to reach ambient temperature and solidify the wax bead. Tests were conducted with 2% or 20 mg of niacin incorporated within the wax bead providing effective performance.

### Example 4

A modified natural wax and polyethylene glycol polymer wax with appropriate molecular weight and melting point formulation is melted to bring the wax few degrees (˜1-10° C.) above the melting point, to which 100 mg of finely powdered water soluble sildenafil citrate is added to form a mixture. A bead of the mixture is dropped into the tip of a condom on the interior surface and is allowed to reach ambient temperature. The condom is then packaged. A 1% concentration of sildenafil citrate in a 1 gram wax bead will deliver 10 mg of sildenafil citrate.

### Example 5

A mixture of polyethylene glycol polymer waxes with appropriate molecular weight and melting points is melted to bring the wax few degrees (˜1-10° C.) above the melting point, to which 10-100 mg of powdered benzocaine is added to form a mixture. A bead of the mixture is dropped into the tip of a condom on the interior surface and is allowed to reach ambient temperature. The condom is then packaged.

### Example 6

A raw material having a mixture of polyethylene glycol waxes, PEG-6 and PEG-32 is sold under the product name Lanogen 1500 by Clariant, was melted to bring the wax few degrees (˜1-10° C.) above the melting point, to which 1-3% by weight of methyl nicotinate and 0.001 to 0.005% by weight of menthol crystal (menthol L) were added to form a mixture. A 0.5 to 2 gram bead of the mixture was dropped into the tip of a condom on the interior surface and was allowed to reach ambient temperature. A silicone oil was applied to the exterior surface of the condom in an amount in the range of 0.2 to 1 gram.

### Example 7

A product having a mixture of polyethylene glycol waxes, PEG-6 and PEG-32 is sold under the product name Lanogen 1500 by Clariant, was melted to bring the wax few degrees (11-10° C.) above the melting point, to which 1-3% by weight of methyl nicotinate and 2-5% by weight of liquid menthol lactate were added to form a mixture. A 0.5 to 2 gram bead of the mixture was dropped into the tip of a condom on the interior surface and was allowed to reach ambient temperature. A silicone oil was applied to the exterior surface of the condom in an amount in the range of 0.2 to 1 gram.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A condom comprising: an elastomeric layer and a low melt wax bead which comprises an active ingredient mixed therein, wherein the bead is affixed on an interior surface of the elastomeric layer in a tip of the condom.

2. The condom of claim 1, wherein the bead softens in the presence of body heat thereby substantially exposing the active ingredient.

3. The condom of claim 1, wherein an exterior surface of the elastomeric layer is substantially free of the active ingredient.

4. The condom of claim 1, wherein an exterior surface of the elastomeric layer further comprises a lubricant that is compatible and immiscible with the low melt wax bead.

5. The condom of claim 1, wherein the interior surface of the elastomeric layer further comprises a lubricant that is compatible and immiscible with the low melt wax bead.

6. The condom of claim 1, wherein the active ingredient comprises a vasodilator.

7. The condom of claim 6, wherein the vasodilator comprises a niacin compound.

8. The condom of claim 6, wherein the vasodilator comprises a sildenafil citrate compound.

9. The condom of claim 1, wherein the bead has a melting point in the range of 25-40° C.

10. The condom of claim 1, wherein the bead comprises a natural wax selected from the group consisting of montan, carnauba, bees wax, bayberry-myrtle, candelialla, caranday, castor bean, asparto-grass, Japan, ouricury, retamo-ceri, mimbi, schlack, spermaceti, sugar-cane, and wool lanolin the melting point of which is reduced to 25° C. to 40° C. by melting with a low melting wax.

11. The condom of claim 1, wherein the bead comprises one or more polyethylene glycol polymers having an overall molecular weight in the range of 600 to 1500 and having a melting point in the range of 25° C. to 40° C.

12. The condom of claim 1, wherein the elastomeric layer comprises natural rubber latex, synthetic polyisoprene, guayule, polyurethane, copolymers thereof, or combinations thereof.

13. The condom of claim 4, wherein the lubricant on the exterior surface of the elastomeric layer further comprises at least one ingredient selected from the group consisting of: L-arginine, a menthol compound, a prostaglandin compound, and an astringent.

14. A condom comprising an elastomeric layer and a low melt wax bead which comprises an erectogenic ingredient, a spermicide, a desensitizing compound, or combinations thereof mixed therein, the bead comprising a modified natural wax or a polyethylene glycol polymer wax with a melting point in the range of 25° C. to 40° C., wherein the bead is affixed on the interior surface of the elastomeric layer in the tip of the condom and wherein the exterior surface of the elastomeric layer is substantially free of the erectogenic ingredient, the spermicide, and the desensitizing compound.

15. The condom of claim 14, wherein the erectogenic ingredient in the wax bead is a niacin compound that is present in an amount in the range of 1-3% by weight.

16. The condom of claim 14, wherein the erectogenic ingredient in the wax bead is sildenafil citrate and is present in an amount in the range of 10 to about 100 mg.

17. The condom of claim 14 further comprising a lubricant that is compatible and immiscible with the bead on the exterior surface, the interior surface, or both.

18. The condom of claim 14, wherein the wax bead further comprises menthol, menthyl lactate, derivatives thereof, or precursors thereof.

19. The condom of claim 14, wherein the bead has a melting point in the range of 25° C.-40° C.

20. A method for manufacturing a condom, the method comprising the steps of:
melting a low melt wax;
mixing an active ingredient in the form of a liquid or a fine dispersion of water soluble powder with the low melt wax to form a substantially uniform mixture;
providing the condom;
dropping a bead of the mixture on the interior surface and in the tip of the condom;
permitting the bead to reach ambient temperature and solidify the wax bead such that the bead is localized on the interior surface and in the tip of the condom; and
packaging the condom.
